(19) United States
(12) Patent Application Publication
(10) Pub. No.: US 2025/0295484 A1
(43) Pub. Date: Sep. 25, 2025

(54) DEVICE FOR TREATMENT OF HEMATOMAS

(71) Applicant: Craig D. ALDINGER, Pittsburgh, PA (US)

(72) Inventor: Craig D. ALDINGER, Pittsburgh, PA (US)

(21) Appl. No.: 18/861,518

(22) PCT Filed: May 5, 2023
(86) PCT No.: PCT/US2023/021190

§ 371 Date: Oct. 29, 2024

Related U.S. Application Data
(60) Provisional application No. 63/364,309, filed on May 6, 2022.

(51) Int. Cl. A61D 1/00

(52) U.S. Cl. CPC A61D 1/00

(57) ABSTRACT

A catheter having a shaft having an elongated body, a first end, a second end, an inner diameter, an outer diameter and a first port; a stylet adapted to fit slidably within the inner diameter of the hollow shaft and adapted to be removed from the hollow shaft from the first port; and a second port adapted to receive a needle and in fluid communication with a balloon positioned along the outer diameter of the hollow shaft; wherein the second end is adapted to receive a fluid and the first end is adapted to drain the fluid.

### CROSS-REFERENCE TO RELATED APPLICATIONS

This application claims the benefit of priority to U.S. Provisional Patent Application Ser. No. 63/364,309, filed on May 6, 2022, entitled DEVICE FOR TREATMENT OF AURAL HEMATOMAS, which is expressly incorporated herein by reference in its entirety.

### TECHNICAL FIELD

The present disclosure relates to medical catheters and, more particularly, but not exclusively, to catheters designed for veterinary applications.

### BACKGROUND

A hematoma is a pool of mostly clotted blood that forms in an organ, tissue, or body space. A hematoma is usually caused by a broken blood vessel that was damaged by surgery or an injury. Hematomas may occur anywhere in the body. Aural hematomas are blood filled swellings on the pinna, or ear lobe, formed secondary to traumatic rupture of the capillaries, thus causing separation of the cartilage and skin.

As an example, conventional approaches to addressing hematomas in veterinary applications often lead to temporary relief, and recurrence is likely within minutes to hours. Surgery may be cost prohibitive, requires full sedation, extensive time, and demands considerable post-operative care.

### SUMMARY

The present disclosure describes a catheter for the treatment of hematomas that includes a hollow shaft comprising an elongated body, a first end, a second end, an inner diameter, an outer diameter and a first port; a stylet adapted to fit slidably within the inner diameter of the hollow shaft and adapted to be removed from the hollow shaft from the first port; and a second port adapted to receive a needle and in fluid communication with a balloon positioned along the outer diameter of the hollow shaft; wherein the second end is adapted to receive a fluid and the first end is adapted to drain the fluid.

The present disclosure describes a method of treating a hematoma and a kit comprising the catheter of the present disclosure.

### BRIEF DESCRIPTION OF THE DRAWINGS

It is to be understood that both the foregoing summary and the following drawings and detailed description may be exemplary and may not be restrictive of the aspects of the present disclosure as claimed. Certain details may be set forth in order to provide a better understanding of various features, aspects, and advantages of the invention. However, one skilled in the art will understand that these features, aspects, and advantages may be practiced without these details. In other instances, well-known structures, methods, and/or processes associated with methods of practicing the various features, aspects, and advantages may not be shown or described in detail to avoid unnecessarily obscuring descriptions of other details of the invention.

FIG. 1 shows a catheter of the present disclosure.

FIG. 2 shows a catheter of the present disclosure inserted into a hematoma.

FIG. 3 shows a catheter having a stylet of the present disclosure placed within a hematoma according to a method of the present disclosure.

FIG. 4 shows a syringe inflating the balloon of the catheter of the present disclosure according to a method of the present disclosure.

FIG. 5 shows the attachment of the cap and the draining bag to the first port of the catheter of the present disclosure.

FIG. 6 shows the catheter of the present disclosure placed within a hematoma according to a method of the present disclosure, wherein the hematoma is drained of fluid.

FIG. 7 shows a catheter of the present disclosure.

### DETAILED DESCRIPTION

In the following description, certain details are set forth in order to provide a better understanding of various embodiments of the methods disclosed herein. However, one skilled in the art will understand that these embodiments may be practiced without these details and/or in the absence of any details not described herein. In other instances, well-known structures, methods, and/or techniques associated with methods of practicing the various embodiments may not be shown or described in detail to avoid unnecessarily obscuring descriptions of other details of the various embodiments.

While particular aspects have been illustrated and described, it would be obvious to those skilled in the art that various other changes and modifications may be made without departing from the spirit and scope of the invention. Those skilled in the art will recognize or be able to ascertain using no more than routine experimentation, numerous equivalents to the specific apparatuses and methods described herein, including alternatives, variants, additions, deletions, modifications and substitutions. This application is intended to cover all such changes and modifications that are within the scope of this application.

The present disclosure provides devices and methods to treat hematomas in an affordable and convenient manner that may have a decreased recurrence rate when compared to conventional treatments.

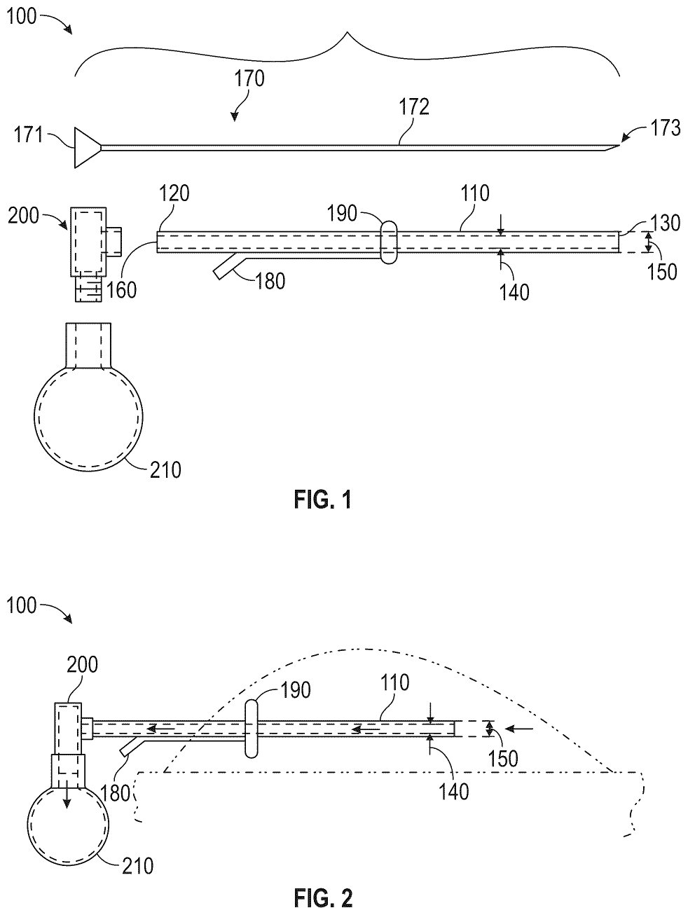

The present disclosure describes a catheter 100 that includes a hollow shaft 110 having a first end 120, a second end 130, an inner diameter 140, an outer diameter 150, and a first port 160 (FIG. 1). The first end 120 may be adapted to drain a fluid into a receptacle 210 attached to a cap 200 at the first port 160, and the second end 130 may be adapted to be inserted into a hematoma to receive the fluid (FIG. 2).

The catheter 100 may include a stylet 170 adapted to fit slidably within the inner diameter 140 of the hollow shaft 110 at the first end 120 (FIG. 1). The stylet 170 may be removed from the hollow shaft 110 from the first port 160. The stylet 170 may include plastic, malleable aluminum, stainless steel, sterile aluminum, the like, and any combination thereof. The length of the stylet 170 may be greater than the length of the hollow shaft 110. As shown in FIG. 1, the stylet 170 may include a first end 171 that includes a handle, a beveled probe 172 having a length and a second end 173 that includes a point. The handle may include a plastic head. The stylet 170 may be used for insertion of the catheter 100 and may be discarded once the catheter 100 is inserted in a hematoma. Once the stylet 170 is removed, the first port 160 may be adapted to receive a cap 200 adapted to receive a receptacle 210. The cap 200 may be screwed onto the first port 160. Accordingly, the first port 160 and cap 200 may include threads to allow for attachment.

The catheter 100 may include a second port 180 adapted to receive an inflationary device 191. An inflationary device 191 may include a needle in fluid communication with a balloon 190 positioned along the outer diameter 150 of the hollow shaft 110. The needle may be of any length or gauge capable of injecting air to inflate the balloon 190. Thus, the balloon 190 may be adapted to be inflated by air applied by the needle and to expand sufficiently to hold the catheter 100 in a treatment position in a hematoma. The needle may be in fluid communication with a syringe, wherein the plunger of a syringe may be pushed inside the barrel of the syringe to expel the air through the needle and into the balloon 190. The syringe may include a pre-measured volume of air necessary to inflate the balloon. While a needle and syringe have been described, other inflationary devices 191, including, but not limited to, needless inflationary devices, are possible and within the scope of the present disclosure. An inflationary device 191 may include a bulb with a one-way valve having a pre-measured volume of air. When providing compression to the bulb, the air may be expelled into the balloon 190. When compression is stopped, the valve closes to prevent release of air from the balloon 190. In order to remove the air from the balloon 190, the valve may be removed. An inflationary device 191 may include a syringe without a needle, wherein the syringe may attach directly to the second port 180 and inflate the balloon 190. To remove the air from the balloon 190 using a needle, the needle in fluid communication with a syringe may be inserted into the second port 180, and the plunger of a syringe may be pulled to from the barrel of the syringe to draw the air into the barrel and remove the air from the balloon 190. The balloon 190 allows for secure placement of the catheter 100 in the interior of a hematoma.

The second port 180 may include an injection cap capable of receiving the needle. The injection cap may include a septum that forms a barrier between the inside of the balloon 190 and the environment. The septum may include rubber or an elastomeric compound capable of maintaining a seal when a needle is inserted and/or removed. The injection cap may include a film that self-seals when the needle is removed.

The second end 130 may be adapted to receive a fluid. As used herein, fluid may include blood, cerebrospinal fluid, urine, interstitial fluid, extracellular fluid, pericardial fluid, lymph, mucus, transcellular fluid, plasma, bile, amniotic fluid, peritoneal fluid, synovial fluid, or any other fluid that may be present within an animal. The fluid may travel from the second end 130 through the inner diameter 140 of the hollow shaft 110 to the first end 120, wherein the first end 120 may be adapted to drain the fluid.

The catheter may include a cap 200, wherein the cap may be adapted to form a seal on the outer diameter 150 of the hollow shaft 110 at the first end 120 in fluid communication with a receptacle 210. The cap 200 may include a valve to control flow of the fluid from the hollow shaft 110 to the receptacle 210. The receptacle 210 may include a draining bag or a draining line connected to the cap 200 and a collection bag. The use of a receptacle 210 such as a draining bag or a collection bag creates a closed drainage system that may be removed with ease at the end of or during treatment.

The receptacle 210 may include a fluid volume of 1 mL to 20 mL, such as, without limitation, 1, 2, 3, 4, 5, 6, 7, 8, 9, 10, 11, 12, 13, 14, 15, 16, 17, 18, 19, and 20 mL. Any combination of lower and upper limits may define the fluid volume of the receptacle 210, such as, 1 mL to 20 mL, including, without limitation, 1 mL to 5 mL, 5 mL to 10 mL, 10 mL to 15 mL, and 15 mL to 20 mL. If the volume of fluid to be drained is less than 1 mL, a receptacle 210 may not be necessary, and the fluid may drain from the catheter on its own. A fluid volume greater than 20 mL may have a weight that is too high, necessitating the replacement of the receptacle 210 to avoid damage to anatomical structures or the catheter. However, if the receptacle 210 includes a draining bag, the fluid volume may include up to 5 L, including, but not limited to, 0.5 L, 1 L, 2 L, 3 L, 4 L, and 5 L. While a receptacle 210 having a range of fluid volumes has been described, other volumes and receptacles 210 are possible and within the scope of the present disclosure dependent on the animal being treated. Accordingly, the present disclosure provides a catheter system wherein more than one receptacle 210 may be necessary in the treatment process to collect all of the fluid present in a hematoma.

It may be useful to provide a catheter in an open system. In an open system, the catheter does not include a cap 200 or a receptacle 210 such as a drainage bag or collection bag. The cap 200 and/or receptacle 210 may be removed from the catheter to convert the catheter from a closed drainage system to an open drainage system. An absorbent pad may be provided for an open drainage system to absorb fluid. The absorbent pad may be attached to a collar such as an Elizabethan collar or any other device capable of holding the absorbent pad in place, including, but not limited to, tape, a clip, and/or the like.

While an open system is possible and within the scope of the present disclosure, a closed system may also be used. A closed system can result in less fluid clean up and provides a decreased likelihood for the development of an infection.

The hollow shaft 110 may have a length of at least 3 mm, such as, without limitation, 3, 5, 10, 15, 20, 25, 30, 35, 40, 45, 50, 55, 60, 65, 70, 75, 80, 90, 100, 110, 120, 130, 140, 150, and 160 mm. The hollow shaft 110 may have a length of no more than 160 mm, such as not more than 150, 140, 130, 120, 110, 100, 90, 80, 75, 70, 65, 60, 55, 50, 45, 40, 35, 30, 25, 20, 15, 10, 5, and 3 mm. Any combination of lower and upper limits may define the length of the hollow shaft 110, such as, 3 mm to 160 mm, including, without limitation, 3 mm to 25 mm, 25 mm to 50 mm, 50 mm to 75 mm, 75 mm to 100 mm, 100 mm to 125 mm, and 125 mm to 160 mm.

The catheter may have a gauge of at least 14, such as, without limitation, 14, 15, 16, 17, 18, 19, 20, 21, and 22. The catheter may have a gauge of no more than 22, such as not more than 21, 20, 19, 18, 17, 16, 15, and 14. Any combination of lower and upper limits may define the gauge, such as, 14 to 16, 16 to 18, 18 to 20, and 20 to 22.

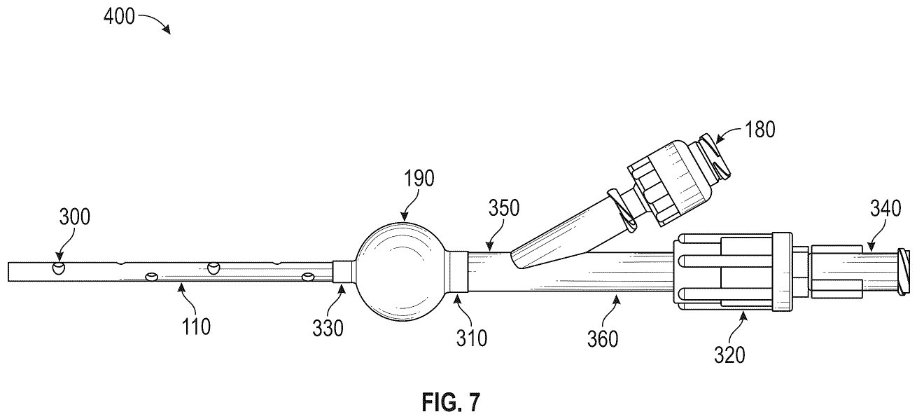

The catheter 400 may include at least one fenestration 300 along the hollow shaft 110 (FIG. 7). The at least one fenestration 300 may prevent obstruction during drainage of fluid by allowing a low-pressure drainage source. Fenestrations 300 may be staggered evenly along the length and circumference of the hollow shaft 110. The distance from each fenestration 300 may be at least 1.0 mm, such as, without limitation, 1.5, 2.0, 2.5, 3.0, 3.5, 4.0, 4.5, and at least 5.0 mm. The distance from each fenestration 300 may be no more than 5.0 mm, such as not more than 4.5, 4.0, 3.5, 3.0, 2.5, 2.0, 1.5, and 1.0 mm. Any combination of lower and upper limits may define the distance from each fenestration 300, such as 1.0 mm to 1.5 mm, 1.5 mm to 2.0 mm, 2.0 mm to 2.5 mm, 2.5 mm to 3.0 mm, 3.0 mm to 3.5 mm, 3.5 mm to 4.0 mm, 4.0 mm to 4.5 mm, and 4.5 mm to 5.0 mm.

The outer diameter 150 of the hollow shaft 110 may have a diameter of at least 0.1 mm, such as, without limitation, 0.1, 0.2, 0.3, 0.4, 0.5, 0.6, 0.7, 0.8, 0.9, and 1.0 mm. The outer diameter 150 may have a diameter of no more than 1.0 mm, such as not more than 0.9, 0.8, 0.7, 0.6, 0.5, 0.4, 0.3, 0.2, and 0.1 mm. Any combination of lower and upper limits may define the diameter of the outer diameter 150, such as 0.1 mm to 0.2 mm, 0.2 mm to 0.3 mm, 0.3 mm to 0.4 mm, 0.4 mm to 0.5 mm, 0.5 mm to 0.6 mm, 0.6 mm to 0.7 mm, 0.7 mm to 0.8 mm, 0.8 mm to 0.9 mm, and 0.9 mm to 1.0 mm.

The inner diameter 140 of the hollow shaft 110 may be smaller than the outer diameter 150. The inner diameter 140 may have a diameter of at least 0.05 mm, such as, without limitation, 0.1, 0.2, 0.3, 0.4, 0.5, 0.6, 0.7, 0.8, 0.9, 0.95 mm The inner diameter 140 may have a diameter of no more than 0.95 mm, such as not more than 0.9, 0.8, 0.7, 0.6, 0.5, 0.4, 0.3, 0.2, 0.1, and 0.05 mm. Any combination of lower and upper limits may define the diameter of the inner diameter 140, such as 0.05 mm to 0.1 mm, 0.1 mm to 0.2 mm, 0.2 mm to 0.3 mm, 0.3 mm to 0.4 mm, 0.4 mm to 0.5 mm, 0.5 mm to 0.6 mm, 0.6 mm to 0.7 mm, 0.7 mm to 0.8 mm, 0.8 mm to 0.9 mm, and 0.9 mm to 0.95 mm.

The stylet 170 may have an outer diameter of 0.05 mm to 0.95 mm. The stylet 170 may have an outer diameter of at least 0.05 mm, such as, without limitation, 0.1, 0.2, 0.3, 0.4, 0.5, 0.6, 0.7, 0.8, 0.9, and 0.95 mm. The stylet 170 may have an outer diameter of no more than 0.95 mm, such as not more than 0.9, 0.8, 0.7, 0.6, 0.5, 0.4, 0.3, 0.2, 0.1, and 0.05 mm. Any combination of lower and upper limits may define the outer diameter of the stylet 170, such as 0.05 mm to 0.1 mm, 0.1 mm to 0.2 mm, 0.2 mm to 0.3 mm, 0.3 mm to 0.4 mm, 0.4 mm to 0.5 mm, 0.5 mm to 0.6 mm, 0.6 mm to 0.7 mm, 0.7 mm to 0.8 mm, 0.8 mm to 0.9 mm, and 0.9 mm to 0.95 mm.

The stylet 170 may have an inner diameter of 0.04 to 0.9 mm. The stylet 170 may have an inner diameter of 0.04 to 0.9 mm. The stylet 170 may have an inner diameter of at least 0.04 mm, such as, without limitation, 0.05, 0.06, 0.07, 0.08, 0.09, 0.1, 0.2, 0.3, 0.4, 0.5, 0.6, 0.7, 0.8, and 0.9 mm. The stylet 170 may have an inner diameter of no more than 0.90 mm, such as not more than 0.9, 0.8, 0.7, 0.6, 0.5, 0.4, 0.3, 0.2, 0.1, 0.09, 0.08, 0.07, 0.06, 0.05, and 0.04 mm. Any combination of lower and upper limits may define the inner diameter of the stylet 170, such as 0.04 mm to 0.06 mm, 0.06 mm to 0.1 mm, 0.1 mm to 0.2 mm, 0.2 mm to 0.3 mm, 0.3 mm to 0.4 mm, 0.4 mm to 0.5 mm, 0.5 mm to 0.6 mm, 0.6 mm to 0.7 mm, 0.7 mm to 0.8 mm, and 0.8 mm to 0.9 mm.

The catheter may be made of polyurethane, silicone, stainless steel, metal, polyvinyl chloride, rubber, latex, plastic such as polyethylene, polypropylene, and/or rigid thermoplastics, and the like, and combinations thereof.

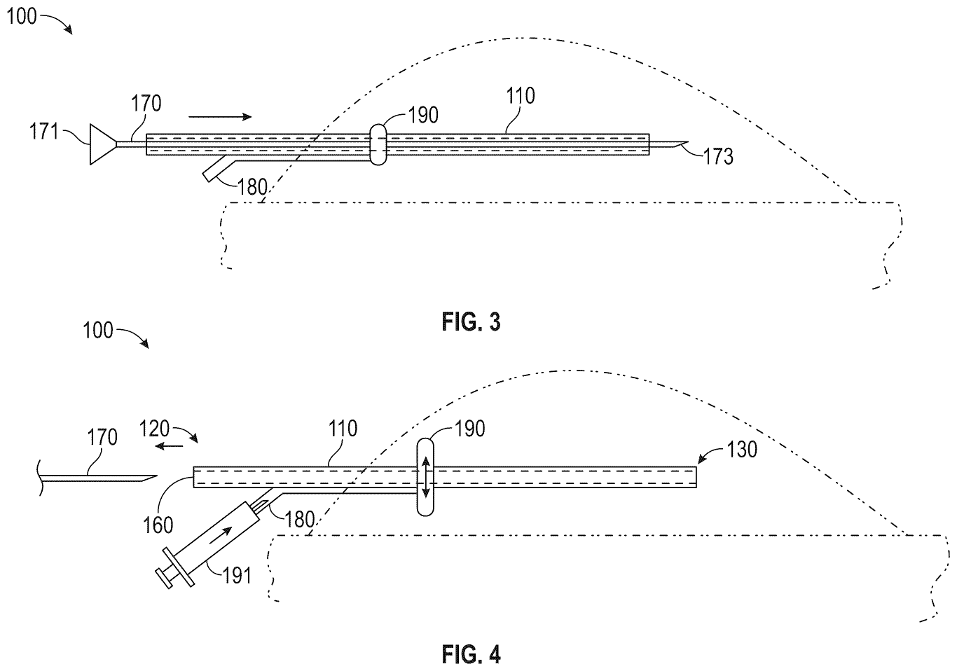

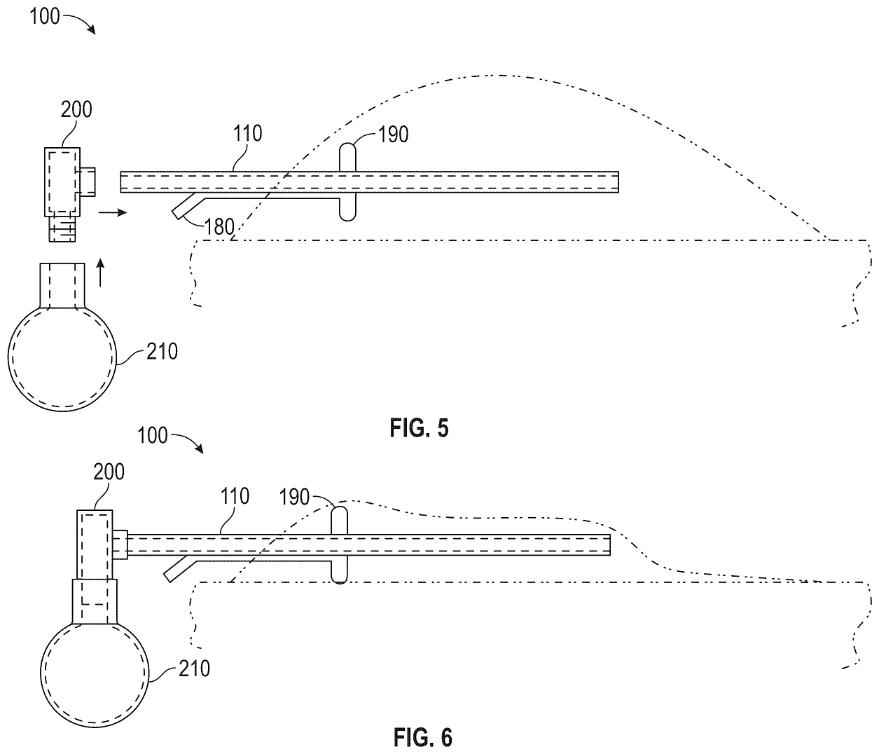

The present disclosure provides a method of treating a hematoma. The method includes inserting the catheter 100 of the present disclosure into a hematoma (FIG. 3). The stylet 170 may be removed from the first port 160 and discarded (FIG. 4). Once the stylet 170 is removed, the balloon 190 may be inflated using an inflationary device according to the present disclosure (FIG. 4). A cap 200 with a receptacle 210 may be attached to receive fluid from the catheter 100, allowing for continuous drainage of the fluid in a closed system (FIG. 5). Continuous drainage of the fluid prevents the hematoma from reforming and allows for the hematoma lumen or “pocket” to heal or scar down. The cap 200 with the receptacle 210 may be easily removed, discarded, and replaced to allow for continuous drainage of the hematoma.

The catheter 100 may be inserted into a hematoma to provide immediate and long-term relief by allowing the fluid accumulation in the hematoma to drain according to the present disclosure. After inflation of the balloon 190, the catheter 100 may remain in place for an extended period of time, including, but not limited to, from one day to three weeks. For example, the catheter 100 may remain until the hematoma has depressed and/or the desired amount of fluid has been drained (FIG. 6).

The overall design of the catheter may be minimal in terms of space occupied within the lumen of the hematoma, allowing for more complete healing prior to catheter removal, an atraumatic removal process, and a decreased likelihood of hematoma recurrence.

The present disclosure provides a kit that includes a catheter according to the present disclosure, at least one inflationary and/or deflationary device, at least one catheter cap, and at least one receptacle. All components of the kit may be sterile. A user may open the prepackaged kit following an antiseptic scrub of the region that includes a hematoma. The catheter (100 or 400) that includes the stylet 170 may be inserted into the most dependent aspect of the hematoma. Once the catheter is inserted, the stylet 170 may be removed and an inflationary device 191 that includes a syringe having a pre-measured volume of air may be inserted into the second port 180 via a needle, and the plunger of the syringe may be depressed completely to inflate the balloon 190. The cap 200 and receptacle 210 may then be attached to collect the fluid. At least two receptacles 210 and caps 200 may be provided in a kit. The cap 200 and receptacle 210 may be replaced when volume capacity of the receptacle 210 has been reached.

Removal of the catheter (100 or 400) may include inserting a deflationary and/or inflationary device into the second port 180, withdrawing the air from the balloon 190, and removing the catheter 100 from the drained hematoma. The catheter (100 or 400) may then be discarded. If complete resolution of the hematoma has not been reached, a second catheter (100 or 400) may be inserted as previously described.

The catheter may remain inserted into a hematoma for at least one day, such as, without limitation, 2, 3, 4, 5, 6, 7, 8, 9, 10, 11, 12, 13, 14, 15, 16, 17, 18, 19, 20, and 21 days. The catheter may be removed from a drained hematoma after no more than 21 days, such as not more than 20, 19, 18, 17, 16, 15, 14, 13, 12, 11, 10, 9, 8, 7, 6, 5, 4, 3, 2, and 1 day. Any combination of lower and upper limits may define the duration of time a catheter is inserted into a hematoma, including, but not limited to, 1 day to 3 days, 3 days to 6 days, 6 days to 9 days, 9 days to 12 days, 12 days to 15 days, 15 days to 18 days, and 18 days to 21 days.

The catheter may remain inserted into a hematoma for less than one day wherein it is desirable to provide complete or partial drainage of a hematoma or surgical cavity within hours.

The presently disclosed catheter may be used during surgeries in which a space is formed, such as mass removals. Insertion of the catheter following surgery may allow for continuous postoperative fluid drainage in a closed system. As such, the present disclosure may be an alternative to the use of conventional drains such as a Penrose or Jackson Pratt drain.

The presently disclosed catheter 400 may include a Y connecter 350 having a drainage shaft 360 and a second port 180 (FIG. 7). The drainage shaft 360 may be adapted to receive a lock 320, such as a luer lock, adapted to receive a fitting 340, wherein the fitting 340 may aid in the removal of the stylet 170. The lock 320 may provide a secure connection between the catheter 400 and a receptacle 210 to maintain a closed system and reduce the chance of fluid leakage or infection. The Y connector 350 may be attached to the balloon 190 via an adhesive bond, wherein adhesives may comprise any suitable UV and LED curing adhesive, including, but not limited to, acrylic-based adhesives, cyanoacrylate-based adhesives, and the like. The hollow shaft 110 may be attached to the balloon 190 via a thermal bond 330. A thermal bond 330 may include any attachment resulting from the use heat. The catheter 400 of the present disclosure may further comprise bonds as a result of laser bonding.

Unlike conventional approaches, the presently disclosed catheter is minimally invasive, does not require a user to have special training, does not require sedation to insert, and may be inserted in under one minute.

### Definitions

In this disclosure, certain terms are used which shall have the meanings set forth as follows.

Various aspects of the methods may be described and illustrated with reference to one or more exemplary implementations. As used herein, the term “exemplary” means “serving as an example, instance, or illustration,” and should not necessarily be construed as preferred or advantageous over other variations of the devices, systems, or methods disclosed herein. “Optional” or “optionally” means that the subsequently described event or circumstance may or may not occur, and that the description includes instances where the implementation occurs and instances where it does not.

As used herein, the term “animal” refers to a dog, cat, bird, rabbit, guinea pig, turtle, hamster, horse, or any other animal capable of developing a hematoma or receiving surgical treatment.

As used herein, a “user” may include any individual, including, but not limited to, an animal owner, a veterinarian, a nurse, a physician, a physician assistant, a veterinary technician, and/or any other individual capable of placing a catheter in a hematoma.

The terminology used herein is for the purpose of describing particular examples only and is not intended to be limiting. As used herein, the singular forms “a”, “an”, and “the” may be intended to include the plural forms as well, unless the context clearly dictates otherwise. As example, “a” receptacle may include multiple receptacles, and the like.

As used herein, the term “and/or” includes any and all combinations of one or more of the associated listed items. Likewise, as used herein, a term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances.

Words such as “then,” “next,” etc. are not intended to limit the order of the steps; these words are simply used to guide the reader through the description of the methods.

The terms “comprises”, “comprising”, “including”, “having”, and “characterized by”, may be inclusive and therefore specify the presence of stated features, elements, compositions, steps, integers, operations, and/or components, but do not preclude the presence or addition of one or more other features, integers, steps, operations, elements, components, and/or groups thereof. Although these open-ended terms may be to be understood as a non-restrictive term used to describe and claim various aspects set forth herein, in certain aspects, the term may alternatively be understood to instead be a more limiting and restrictive term, such as “consisting of” or “consisting essentially of.” Thus, for any given embodiment reciting compositions, materials, components, elements, features, integers, operations, and/or process steps, described herein also specifically includes embodiments consisting of, or consisting essentially of, such recited compositions, materials, components, elements, features, integers, operations, and/or process steps. In the case of “consisting of”, the alternative embodiment excludes any additional compositions, materials, components, elements, features, integers, operations, and/or process steps, while in the case of “consisting essentially of”, any additional compositions, materials, components, elements, features, integers, operations, and/or process steps that materially affect the basic and novel characteristics may be excluded from such an embodiment, but any compositions, materials, components, elements, features, integers, operations, and/or process steps that do not materially affect the basic and novel characteristics may be included in the embodiment.

Any method steps, processes, and operations described herein may not be construed as necessarily requiring their performance in the particular order discussed or illustrated, unless specifically identified as an order of performance. It is also understood that additional or alternative steps may be employed, unless otherwise indicated.

In addition, features described with respect to certain example embodiments may be combined in or with various other example embodiments in any permutational or combinatory manner. Different aspects or elements of example embodiments, as disclosed herein, may be combined in a similar manner. The term “combination”, “combinatory,” or “combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included may be combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

While specific aspects of the disclosure have been provided hereinabove, the disclosure may, however, be embodied in many different forms and should not be construed as necessarily being limited to only the embodiments disclosed herein. Rather, these embodiments may be provided so that this disclosure is thorough and complete, and fully conveys various concepts of this disclosure to skilled artisans.

All numerical quantities stated herein may be approximate, unless stated otherwise. Accordingly, the term “about” may be inferred when not expressly stated. The numerical quantities disclosed herein may be to be understood as not being strictly limited to the exact numerical values recited. Instead, unless stated otherwise, each numerical value stated herein is intended to mean both the recited value and a functionally equivalent range surrounding that value. At the very least, and not as an attempt to limit the application of the doctrine of equivalents to the scope of the claims, each numerical value should at least be construed in light of the number of reported significant digits and by applying ordinary rounding processes. Typical exemplary degrees of error may be within 20%, 10%, or 5% of a given value or range of values. Alternatively, the term “about” refers to values within an order of magnitude, potentially within 5-fold or 2-fold of a given value. Notwithstanding the approximations of numerical quantities stated herein, the numerical quantities described in specific examples of actual measured values may be reported as precisely as possible. Any numerical values, however, inherently contain certain errors necessarily resulting from the standard deviation found in their respective testing measurements.

All numerical ranges stated herein include all sub-ranges subsumed therein. For example, a range of “1 to 10” or “1-10” is intended to include all sub-ranges between and including the recited minimum value of 1 and the recited maximum value of 10 because the disclosed numerical ranges may be continuous and include every value between the minimum and maximum values. Any maximum numerical limitation recited herein is intended to include all lower numerical limitations. Any minimum numerical limitation recited herein is intended to include all higher numerical limitations.

Features or functionality described with respect to certain example embodiments may be combined and sub-combined in and/or with various other example embodiments. Also, different aspects and/or elements of example embodiments, as disclosed herein, may be combined and sub-combined in a similar manner as well. Further, some example embodiments, whether individually and/or collectively, may be components of a larger system, wherein other procedures may take precedence over and/or otherwise modify their application. Additionally, a number of steps may be required before, after, and/or concurrently with example embodiments, as disclosed herein. Note that any and/or all methods and/or processes, at least as disclosed herein, may be at least partially performed via at least one entity or actor in any manner.

All documents cited herein may be incorporated herein by reference, but only to the extent that the incorporated material does not conflict with existing definitions, statements, or other documents set forth herein. To the extent that any meaning or definition of a term in this document conflicts with any meaning or definition of the same term in a document incorporated by reference, the meaning or definition assigned to that term in this document shall govern. The citation of any document is not to be construed as an admission that it is prior art with respect to this application.

While particular embodiments have been illustrated and described, it would be obvious to those skilled in the art that various other changes and modifications may be made without departing from the spirit and scope of the invention. Those skilled in the art will recognize or be able to ascertain using no more than routine experimentation, numerous equivalents to the specific apparatuses and methods described herein, including alternatives, variants, additions, deletions, modifications and substitutions. This application including the appended claims is therefore intended to cover all such changes and modifications that may be within the scope of this application.

Unless defined otherwise, all technical and scientific terms used herein have the same meanings as commonly understood by one of ordinary skill in the art.

1. A catheter, comprising:
a hollow shaft comprising an elongated body, a first end, a second end, an inner diameter, an outer diameter and a first port;
a stylet adapted to fit slidably within the inner diameter of the hollow shaft and adapted to be removed from the hollow shaft from the first port; and
a second port adapted to receive a needle and in fluid communication with a balloon positioned along the outer diameter of the hollow shaft;
wherein the second end is adapted to receive a fluid and the first end is adapted to drain the fluid.

2. The catheter of claim 1, comprising a cap adapted to form a seal on the outer diameter of the hollow shaft at the second end and in fluid communication with a receptacle.

3. The catheter of claim 1, wherein the balloon is adapted to be inflated by air applied through the needle.

4. The catheter of claim 3, wherein the balloon is adapted to expand sufficiently to hold the catheter in a treatment position in a hematoma.

5. The catheter of claim 1, comprising a syringe in fluid communication with the needle and adapted to supply air to the balloon.

6. The catheter of claim 1, wherein the stylet comprises a first end comprising a handle and a second end comprising a point.

7. The catheter of claim 1, wherein the outer diameter of the hollow shaft is from 0.1 to 1 mm, such as from 0.2 to 0.9 mm, from 0.3 mm to 0.8 mm or from 0.3 to 0.75 mm.

8. The catheter of claim 7, wherein the inner diameter of the hollow shaft is smaller than the outer diameter and can range from 0.05 to 0.95 mm, such as from 0.1 to 0.95 mm, from 0.2 mm to 0.75 mm or from 0.25 to 0.7 mm.

9. The catheter of claim 1, wherein the stylet has an outer diameter that can range from 0.05 to 0.95 mm, such as from 0.1 to 0.95 mm, from 0.2 mm to 0.75 mm or from 0.25 to 0.7 mm.

10. The catheter of claim 9, wherein the stylet has an inner diameter that can range from 0.04 to 0.9 mm, such as from 0.075 to 0.85 mm, from 0.1 mm to 0.65 mm or from 0.15 to 0.6 mm.

11. The catheter of claim 1, wherein the hollow shaft has a length and the stylet has a length, wherein the length of the stylet is greater than the length of the hollow shaft.

12. The catheter of claim 1, wherein the fluid is provided to a draining bag.

13. The catheter of claim 1, further comprising fenestrations along the hollow shaft.

14. The catheter of claim 1, wherein the catheter comprises polyurethane and/or silicone.

15. The catheter of claim 1, wherein the shaft comprises a length of 3 mm to 160 mm.

16. The catheter of claim 1, wherein the gauge of the catheter is 14 to 22.

17. The catheter of claim 1, wherein the fluid is provided to a receptacle, wherein the receptacle comprises a fluid volume of 1 mL to 20 mL.

18. (canceled)

19. (canceled)

20. A method of treating a hematoma, the method comprising:
inserting into the hematoma a catheter
removing the stylet from the first port of the catheter;
inflating the balloon;
attaching a receptacle adapted to receive the fluid from the catheter;
inserting a syringe into the second port, wherein the syringe is configured to aspirate air from the balloon;
aspirating the air from the balloon; and
removing the catheter from the hematoma.

21. (canceled)

22. (canceled)

23. (canceled)

24. (canceled)

25. The method of claim 20, wherein the method is used in a veterinary application.

26. (canceled)

27. (canceled)

28. (canceled)

29. (canceled)

30. A kit comprising:
a catheter of claim 1;
at least one inflationary and/or deflationary device;
at least one catheter cap; and
at least one receptacle.
